(19) United States
(12) Patent Application Publication
(10) Pub. No.: US 2008/0058736 A1
(43) Pub. Date: Mar. 6, 2008

(54) Sharps container having absorbent pad and method of making the same

(76) Inventor: Piyush J. Reshamwala, Crystal Lake, IL (US)

Correspondence Address: RATNERPRESTIA, P O BOX 980, VALLEY FORGE, PA, 19482-0980, US

(21) Appl. No.: 11/512,470

(22) Filed: Aug. 30, 2006

(51) Int. Cl. A61M 1/00

(52) U.S. Cl. 604/319

(57) ABSTRACT

A medical waste container includes a receptacle for receiving medical waste. In a first aspect of the invention, a container includes a receptacle having a bottom wall and an absorbent pad positioned in the receptacle adjacent the bottom wall. The absorbent pad includes a substrate containing a super absorbent polymer. In a second aspect of the invention, a waste disposal kit for the collection and disposal of medical wastes includes a plurality of receptacles for receiving medical waste and a plurality of absorbent pads. Each absorbent pad is configured for placement in one of the receptacles adjacent a bottom wall in the receptacle.

### FIELD OF THE INVENTION

The present invention relates generally to containers for the storage and disposal of medical wastes, and more particularly to containers having an absorbent pad for capturing free fluids that accumulate within the waste container.

### BACKGROUND OF THE INVENTION

Sharps waste, chemotherapy waste, RCRA hazardous waste and other forms of medical waste are ideally stored in rigid, puncture-resistant containers. In many cases, medical wastes, including but not limited to used sharps and IV bags, contain liquids which can accumulate in the disposal container and leak from the container. The container may leak during a sudden release if the container is not properly sealed, or gradually over time during transport. Therefore, disposal containers often include bag liners and gasketed lids to reduce the potential for leakage of free fluids.

Bag liners, gasketed lids, and other attempts to address the problems associated with liquid-containing medical waste leave room for improvement. Accordingly, there remains a need for an improved container system for use with liquid-containing medical waste.

### SUMMARY OF THE INVENTION

The foregoing drawbacks of medical waste containers are resolved to a large degree by the present invention, which features a container for the collection and disposal of medical waste. In a first aspect of the invention, a medical waste container includes a receptacle for receiving medical waste. The receptacle has a bottom wall and an absorbent pad positioned in the receptacle adjacent the bottom wall. The absorbent pad includes a substrate which may be made of cellulose tissue. The substrate contains a super absorbent polymer (SAP) and pulp or a synthetic material. The medical waste container has a ratio of absorption capacity of the pad to the volume of the container. In one embodiment, the ratio of the absorption capacity of the pad to the volume of the container is at least about 2 percent. In another embodiment, the ratio of the absorption capacity of the pad to the volume of the container is at least about 5 percent. In yet another embodiment, the absorbent pad has an absorbency of at least about 20 cc water/square inch.

The absorbency data provided herein is based on absorbency tests in which tap water was steadily applied to an absorbent pad. Water was slowly poured onto the pad so as to not displace fibers or otherwise disturb the structure of the pad. Under these conditions, absorbency measurements were not found to vary significantly with the rate at which water was applied to the pad. In addition, absorbency measurements were not found to differ significantly when distilled water was poured onto the pad instead of tap water. Therefore, the absorbency data provided herein assumes either tap water or distilled water applied to the pad.

The waste container may use a variety of absorbent pad configurations. In one embodiment, the absorbent pad has a length of about 10 inches or less and a width of about 6 inches or less. In another embodiment, the absorbent pad has a surface area of about 60 square inches or less. The ratio of the surface area of the pad to the surface area of the bottom wall is about 0.2, or less. In yet another embodiment, the pad has a thickness in the dry state of no greater than about 1/16 of an inch.

The absorbent pad may be arranged in the container in various ways. For example, the absorbent pad may be bonded to the bottom wall of the receptacle with an adhesive. The receptacle may include one or more sidewalls extending from the bottom wall, with the pad being spaced from the one or more sidewalls forming a fluid collection moat around the perimeter of the pad. The pad may include a variety of components, such as a tissue layer and a fibrous material contained within the tissue layer.

In a second aspect of the invention, a waste disposal kit for the collection and disposal of medical wastes includes a plurality of receptacles for receiving medical waste and a plurality of absorbent pads. Each absorbent pad includes a substrate and a super absorbent polymer contained within the substrate. In addition, each absorbent pad is configured for placement in one of the receptacles adjacent a bottom wall in the receptacle. In one embodiment, the ratio of the absorption capacity of each pad to the volume of each container is at least about 2 percent. In another embodiment, the ratio of the absorption capacity of each pad to the volume of each container is at least about 5 percent. In yet another embodiment, each absorbent pad has an absorbency of at least about 20 cc water/square inch. The waste containers may use a variety of absorbent pad configurations. In one kit embodiment, the absorbent pads each have a length of about 10 inches or less and a width of about 6 inches or less. In another embodiment, the absorbent pads each have a surface area of about 60 square inches or less. The ratio of the surface area of each pad to the surface area of the bottom wall of each receptacle may be at least about 0.2. Moreover, each absorbent pad may be arranged in the receptacle in various ways. For example, each absorbent pad may be configured to be bonded to the bottom wall of a receptacle with an adhesive. The container may include one or more sidewalls extending from the bottom wall, with the pad being spaced from the one or more sidewalls forming a fluid collection moat around the perimeter of the pad. The pad may include a variety of components, such as a tissue layer and a fibrous material contained within the tissue layer. The waste disposal kit may be packaged in a variety of ways. For example, the kit may include a film package that contains the plurality of absorbent pads. Alternatively, or in addition, the kit may include a casing that encloses the receptacles and pads together.

### BRIEF DESCRIPTION OF THE DRAWINGS

The foregoing summary as well as the following description will be better understood when read in conjunction with the figures in which:

FIG. 1 is an exploded perspective view of a waste container in accordance with the present invention.

FIG. 2 is a top view of a waste container in accordance with the present invention in an assembled condition.

FIG. 3 is a truncated cross section view of the waste container of FIG. 2, taken through line A-A of FIG. 2.

FIG. 4 is a cross section view of an absorbent pad suitable for use in the present invention.

FIG. 5 is a schematic plan view of a packaged kit in accordance with the present invention.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention. A waste disposal container in accordance with the present invention includes a receptacle for receiving and storing medical waste. The receptacle has a bottom wall and one or more side walls that contain a volume of waste. An absorbent body is positioned in the receptacle to capture liquid that leaches or drains from the waste. The absorbent body traps and contains the liquid to minimize the release of the liquid from the container in the event of a container spill or breakage of the container.

According to exemplary embodiments of this invention, an absorbent pad is placed in the bottom of a container to absorb liquid as the liquid accumulates in the container. This has the advantage of preventing liquid leakage before it starts by trapping liquids within the container before the liquid can leak or spill out of the container.

Such an absorbent pad preferably has sufficient absorbent capacity to trap the quantity of liquid associated with some medical waste, which can be considerable. For example, many syringes are known to exhibit a certain volume of dead space, which is the space between the plunger and needle that contains residual liquid after the plunger is fully advanced to release the contents in the syringe. Assuming a typical dead space of 5% common to all wastes placed in the container, and assuming those dead spaces to be occupied by liquid, the desired absorbency would be at least about 5 percent of the volume of the container. For an 8 gallon container, this would be 0.4 gallons, or approximately 1,500 cubic centimeters.

One approach to achieving an increased absorption capacity is to simply increase the amount of surface area of absorbent padding used in the bottom of the container. If the absorbent capacity of a single pad is insufficient, two or more absorbent pads are placed in the container until the bottom of the container is entirely covered or substantially covered with absorbent padding. This approach may have limitations in certain applications, however. For example, adding absorbent material adds to the unit cost of each container. In addition, many containers are molded with bottoms that are not flat. For example, some containers have a generally rectangular shape with rounded corners. These surfaces make it very difficult to cover the bottom surface of the container with absorbent padding, while keeping the padding in a flattened stable condition. If the pad is not stable, the pad could be shifted and bunched into positions that do not properly absorb liquid from the bottom of the container where it accumulates.

The use of multiple pads in a container may require opening and handling multiple pads. Maintenance workers and medical staff, for a variety of reasons, may therefore fail to place all the desired pads in the container. For example, personnel may be inclined to save one or more of the pads under the mistaken assumption that only one pad is required in the container. Therefore, though viable, increasing the number of absorbent pads in the container may have drawbacks that take away from the effectiveness of this option in certain applications.

According to another exemplary embodiment of the invention, the absorbent body features an increased absorption capacity in combination with a reduced overall size relative to the bottom of the receptacle. The reduced size allows the absorbent body to rest flatly on the bottom of the receptacle, with minimal or no voids created between the pad and the container bottom. The reduced size also reduces the unit cost of the container. At the same time, the increased absorption capacity of the pad accommodates the amount of liquid leachate that is typical from dead space syringes, IV bags, and other common medical waste.

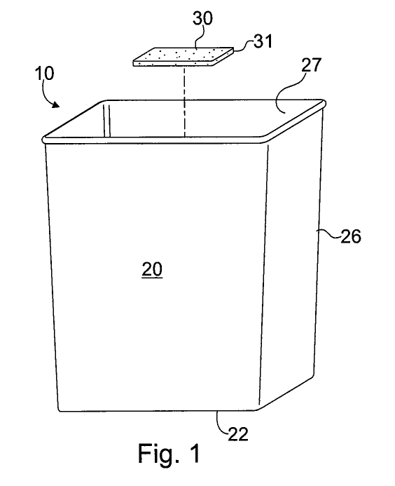

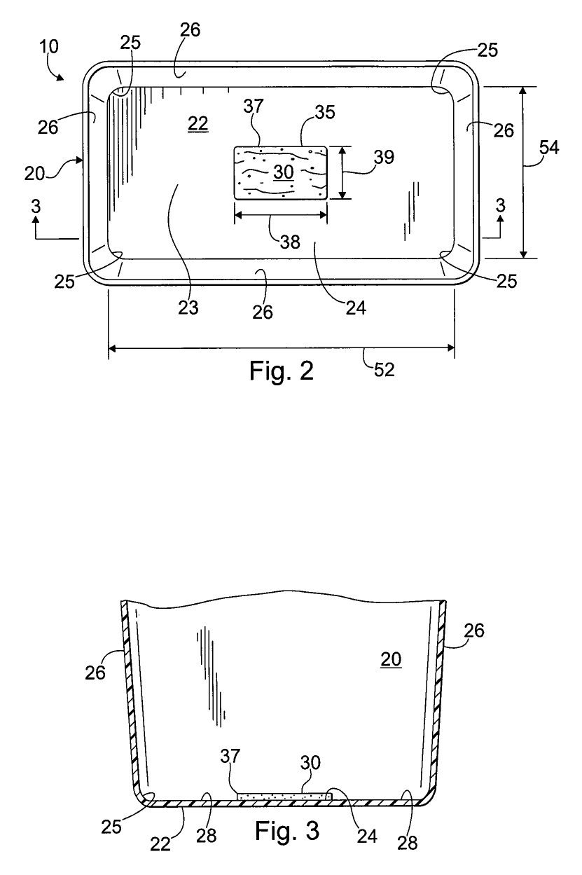

Referring to the drawing Figures generally, and to FIGS. 1 and 2 in particular, a waste disposal container 10 in accordance with the invention will be described in greater detail. The container 10 includes a receptacle 20 having a bottom wall 22. The receptacle of the present invention may be a bucket, bin, drum, canister or the like having variety of shapes and dimensions adapted for the containment of medical waste. The shape and dimensions of the container may depend on such factors as the quantity of waste being stored, the mounting arrangement, and other variables. In FIGS. 1 and 2, the exemplary receptacle 20 is substantially rectangular with four side walls 26 extending from the bottom wall 22 to form an interior space 27. In the. preferred embodiment, the container 10 utilizes receptacles of different standard sizes, for example, 2 gallon, 3 gallon, 8 gallon, 12 gallon and 18 gallon size containers.

Bottom wall 22 has a length 52, a width 54, and an interior surface area 23 having a substantially flat surface contour. As is typical of many waste receptacles, the large majority of interior surface area 23 includes a substantially planar or flat central area 24. The flat central area 24 is surrounded by a non-planar perimeter region, which in FIG. 2 is a curved region 25 extending adjacent to and contiguous with sidewalls 26. Receptacle 20 may be formed of a variety of materials, the selection of which will depend on, among other factors, the nature of the waste being stored and the applicable disposal regulations. In the preferred embodiment, receptacle 20 is formed of polypropylene or other rigid material which provides resistance to puncturing, and is suitable for incineration.

The container of the present invention further includes an absorbent body inserted into the receptacle. Referring to FIGS. 1-3, receptacle 20 contains a rectangular-shaped absorbent pad 30. Absorbent pad 30 rests in a substantially flat position on the flat central area 24 of bottom wall 22. Pad 30 has a length 38 that is less than a length 52 of the bottom wall, and a width 39 that is less than a width 54 of the bottom wall. As such, pad 30 has a perimeter 37 that is offset from the curved region 25 and sidewalls 26, forming a shallow trough or moat 28 between the perimeter 37 of the pad 30 and the sidewalls, as best seen in FIG. 3. For purposes of this description, perimeter 37 refers collectively to the two sides of the pad in FIG. 3 associated with length 38 and the two sides of the pad in FIG. 3 associated with width 39. Perimeter 37 has a total length equal to the lengths of the four sides (i.e. 2(length 38)+2(width 39)).

Pad 30 has a surface area 35 that is confined within the flat central area 24 of the bottom wall 22. In this arrangement, the entire pad 30 rests flatly on the bottom wall 22. For purposes of this description, the surface area 35 of the pad refers to the area of the pad as measured within the four sides that make up the perimeter 37. The surface area is equal to the length 38 times the width 39.

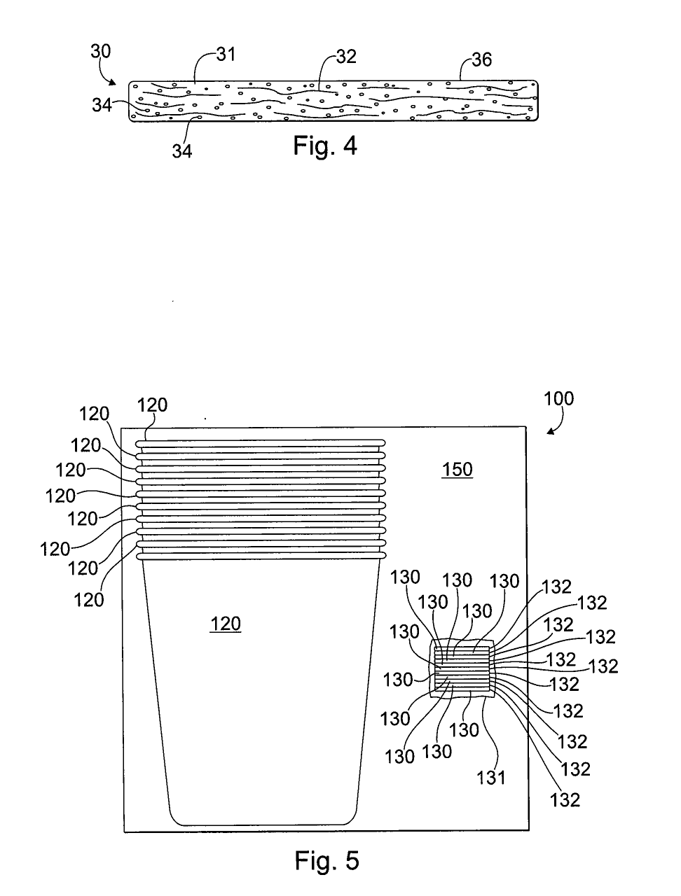

The container 10 has an absorbent body that makes it possible to provide the required absorbency without assuming a large surface area. The absorbent pad has enhanced absorbent properties that reduce the amount of absorbent material required to be placed in the container. Referring to FIG. 4, pad 30 includes a fibrous substrate 32 and a super absorbent polymer (SAP) material 34 contained within the substrate. The SAP material 34 may be provided in a variety of arrangements within the substrate 32. For example, the SAP material 34 may be dispersed throughout the substrate 32 in particle form or fiber form. In addition, the SAP material may bonded to the substrate. A tissue layer 36 surrounds the substrate. In FIG. 4, the SAP material 34 is dispersed throughout the substrate 34 and held within the substrate by an adhesive 31. A variety of materials may be used to form the absorbent pad 30, so long as the pad remains compliant with the characteristics described herein. For example, the fibrous substrate 32 may include cellulose acetate fibers or pulp fibers. An exemplary pad 30 is available from Rayonier under the brand name NOVATHIN.

Absorbent pad 30 has the capacity to absorb a volume of free liquid that is in line with the free liquid volumes observed in medical waste containers. In the preferred embodiment, absorbent pad 30 has an absorbency of at least about 20 cubic centimeters of water per square inch, more preferably an absorbency of at least about 25 cubic centimeters of water per square inch, and still more preferably an absorbency of at least about 30 cubic centimeters of water per square inch.

The absorbency data provided herein was obtained through a series of tests in which water was applied to a pad in its dry state under room temperature and atmospheric pressure. Water was poured onto the pad until the pad reached a point of saturation; that is, the point beyond which any additional liquid poured onto the pad resulted in free liquid accumulating around the pad. The volume of water that was poured up to the saturation point was determined to be the total volume absorbed. The total volume absorbed was then divided by the surface area of the pad to determine the absorbency values in cc/square inch.

Waste disposal container 10 has a ratio of absorption capacity of the pad to the volume of the container. The ratio of the absorption capacity of the pad to the volume of the container is preferably at least about 2 percent, more preferably at least about 3 percent, still more preferably at least about 4 percent, and still more preferably at least about 5 percent.

The waste container may use a variety of absorbent pad configurations. While a variety of pad sizes can be selected within the scope of the present invention, two basic sizes have been found suitable for use with a broad range of container sizes. In one size, absorbent pad 30 has a total surface area of about 18 square inches or less. For example, absorbent pad 30 may have a length of about 6 inches or less and a width of about 3 inches or less. This size has been found most suitable for 2 and 3 gallon containers, although the size may be appropriate for other container sizes as well. In another embodiment, absorbent pad 30 has a surface area of about 60 square inches or less. For example, absorbent pad 30 may have a length of about 10 inches or less and a width of about 6 inches or less. This size has been found most suitable for 8, 12 and 18 gallon containers, although the size may be appropriate for other container sizes as well, including but not limited to 19 gallon and 30 gallon containers. For larger containers, i.e. containers larger than 18 gallons, it may be desirable to provide more than one pad, depending on the application, in order to further increase the absorption of free liquid in the container. The selection of pad size may also be made based on relative surface area. For example, a ratio of surface area of the pad to surface area of the bottom wall of about 0.2 may be used to provide adequate absorption for many categories of waste.

The effective storage capacity of medical waste containers is typically represented by a fill line placed on the container or a label placed onto the container. The fill line is commonly placed three-quarters of the way up the height of the container, measured from the bottom. Medical personnel are instructed not to place any waste in the container once the volume of waste in the container rises to the fill line. In view of this restriction, the effective capacity of a waste container is influenced by the height of any pads that are placed on the bottom of the container, as the pad or pads will occupy some amount of volume that would otherwise be occupied by waste. The presence of highly absorbent SAP has the advantage of providing a large absorption capacity in a pad having a significantly reduced pad thickness. In a preferred embodiment, pad 30 has a very small thickness so as to minimize the amount of space the pad occupies at the bottom of the container. This is advantageous to the user, as it increases the overall storage capacity of the container. Because the pad 30 has a very small thickness as compared to known absorbent pads, the pad has less height and therefore takes up less volume in the container, leaving more room for waste. As a result, more waste can be placed in the container, reducing the frequency of replacement and disposal of waste containers over time. Pad 30 preferably has a thickness in the dry state of about ⅛ inch or less, more preferably about 1/16 inch or less, and even more preferably about 1/32 inch or less.

Bottom wall 22 has a surface area 23 that is adapted to receive absorbent pad 30 in a flat condition with clearance around the perimeter, as noted above. In one exemplary embodiment, absorbent pad 30 is held in place in the central area 24 of bottom wall 22 by a securement means. The securement means may be a fastener integrally molded to the bottom wall of the container, including but not limited to a clip, hook or tab fixture that holds down the edges of pad 30. Referring to FIG. 3, pad 30 is secured to central area 24 of bottom wall 22 by a layer of adhesive 40. In this arrangement, pad 30 is affixed to the bottom wall 22 and resists sliding, buckling or folding in response to contact with waste that is dropped into the receptacle 20. Pad 30 therefore remains flat against the bottom wall 22 of receptacle 20 without voids forming between the pad and the bottom wall 22. Alternatively, the securement means can be eliminated and the pad can simply rest on the container bottom.

The medical waste containers of the present invention may be sold and packaged as a single unit. For example, the waste containers may be sold in a package containing a single receptacle and a single absorbent pad placed in the receptacle. Where applicable, other accessories may be included in the packaging, such as a lid for the receptacle and/or components for mounting the receptacle to a wall or other support structure.

The absorbent pad may be individually wrapped and placed in the packaging with the container. Upon setting up the container, the user would unpack the receptacle and the pad, unwrap the pad, and place the pad into the bottom of the receptacle. If the pad is attachable to the bottom of the receptacle, as for example by an adhesive, the user would apply the unwrapped pad to the bottom of the receptacle with the adhesive side face down onto the bottom of the receptacle.

Absorbent padding is commonly manufactured in rolls and subsequently cut into sheets of specific sizes. Depending on the particular product or end use, the individual absorbent sheets may be packaged in sets of 5, 10, or some other grouping arrangement. Where absorbent pads are supplied from a third party, it has been found beneficial to manufacture and sell the medical container in kits containing multiple receptacles and pads. For example, the medical waste container of the present invention may be sold as a kit containing two or more receptacles and two or more corresponding absorbent pads for placement into the receptacles. A kit arrangement makes it easier to incorporate sets of absorbent pads into the product. Selling containers individually is also an option, but may require opening sets of absorbent pads to separate out individual pads. This separation step may not be desirable as it adds an additional step in the production process.

Referring to FIG. 5, a kit 100 in accordance with the present invention includes ten receptacles 120 and ten absorbent pads 130 packaged together in a casing 150. The receptacles 120 each have an open top, a closed bottom, and a perimeter that tapers inwardly from the open top toward the bottom. The tapered shape of the receptacles 120 allows them to be stacked one inside the other so as to reduce the required volume of casing required to package the kit 100. A variety of packaging arrangements may be utilized to enclose the containers and pads and protect them during shipment. For example, the casing 150 may be a cardboard box with cardboard of paper inserts. In FIG. 5, pads 130 are encased together in a primary film wrap 131. Each pad 130 is further wrapped individually with a secondary film wrap 132, so that when primary film wrap 131 is opened, each pad remains covered and protected from dirt or other contaminants until it's secondary film wrap is opened.

Although the invention is illustrated and described herein with reference to specific embodiments, the invention is not intended to be limited to the details shown. Rather, various modifications may be made in the details within the scope and range of equivalents of the claims and without departing from the invention.

1. A container for the collection and disposal of medical waste, comprising:
A. a receptacle for receiving medical waste, the receptacle having a bottom wall; and
B. an absorbent pad positioned in the receptacle adjacent the bottom wall for absorbing liquid from the medical waste, the absorbent pad including a substrate containing a super absorbent polymer.

2. The container of claim 1, wherein the ratio of the absorption capacity of the pad to the volume of the receptacle is at least about 2 percent.

3. The container of claim 1, wherein the ratio of the absorption capacity of the pad to the volume of the receptacle is at least about 5 percent.

4. The container of claim 1, wherein the absorbent pad has an absorbency of at least about 20 cc water/square inch.

5. The container of claim 1, wherein the absorbent pad has an absorbency of at least about 25 cc water/square inch.

6. The container of claim 1, wherein the absorbent pad has a length of about 10 inches or less and a width of about 6 inches or less.

7. The container of claim 1, wherein the absorbent pad has a surface area of about 60 square inches or less.

8. The container of claim 1, wherein the absorbent pad is bonded to the bottom wall of the receptacle with an adhesive.

9. The container of claim 1, wherein the ratio of the surface area of the pad to the surface area of the bottom wall is at least about 0.2.

10. The container of claim 1, wherein the pad comprises a tissue layer and a fibrous material contained within the tissue layer.

11. The container of claim 1 wherein the receptacle comprises one or more sidewalls extending from the bottom wall, the pad being spaced from said one or more sidewalls forming a fluid collection moat around the perimeter of the pad.

12. A waste disposal kit for the collection and disposal of medical wastes, comprising:
A. a plurality of receptacles for receiving medical waste, each receptacle having a bottom wall; and
B. a plurality of absorbent pads, each including a substrate containing a super absorbent polymer and each configured for placement in one of said receptacles adjacent the bottom wall.

13. The waste disposal kit of claim 12, wherein the ratio of the absorption capacity of each pad to the volume of each receptacle is at least about 2 percent.

14. The waste disposal kit of claim 12, wherein each absorbent pad has an absorbency of at least about 20 cc water/square inch.

15. The waste disposal kit of claim 12, wherein each absorbent pad has an absorbency of at least about 25 cc water/square inch.

16. The waste disposal kit of claim 12, wherein each absorbent pad has a length of about 10 inches or less and a width of about 6 inches or less.

17. The waste disposal kit of claim 12, wherein each absorbent pad has a surface area of about 60 square inches or less.

18. The waste disposal kit of claim 12, wherein each absorbent pad is bonded to the bottom wall of each receptacle with an adhesive.

19. The waste disposal kit of claim 12, wherein each pad comprises a tissue layer and a fibrous material contained within the tissue layer.

20. The waste disposal kit of claim 12 comprising a film package that contains said plurality of absorbent pads, and a casing that encloses said plurality of receptacles and said film package.
